(19) United States
(12) Patent Application Publication
(10) Pub. No.: US 2018/0358131 A1
(43) Pub. Date: Dec. 13, 2018

(54) ASSIGNING PATIENTS TO HOSPITAL BEDS

(71) Applicant: KONINKLIJKE PHILIPS N.V., EINDHOVEN (NL)

(72) Inventors: Douglas Henrique Teodoro, São Paulo (BR); Lucas de Melo Oliveira, Melrose, MA (US)

(21) Appl. No.: 15/775,066

(22) PCT Filed: Nov. 29, 2016
(86) PCT No.: PCT/IB2016/057172

§ 371 Date: May 10, 2018

Related U.S. Application Data
(60) Provisional application No. 62/262,476, filed on Dec. 3, 2015.

(51) Int. Cl. G16H 50/80; G16H 50/30; G16H 40/20

(52) U.S. Cl. CPC G16H 50/80; G16H 40/20; G16H 50/30

(57) ABSTRACT

Methods and apparatus for assigning patients to hospital beds to prevent the spread of healthcare-associated infection (HAI). HAI is a major patient safety problem worldwide, affecting hundreds of millions of individuals each year. By considering probabilities that patients will acquire and/or spread infections, embodiments of the present invention can reduce the spread of HAI by selectively assigning patients to beds within healthcare institutions.

### TECHNICAL FIELD

This invention generally relates to methods and apparatus for assigning people to prevent the spread of disease and, more particularly, to assigning patients to hospital beds to prevent health-care associated infections (HAI).

### BACKGROUND

Patients in healthcare institutions such as hospitals or the like are often at risk of spreading and acquiring illnesses while in the institution. These infections are commonly referred to as healthcare-associated infections (HAI). HAI is a significant problem in healthcare, as approximately 1.7 million Americans develop HAIs annually and 99,000 die from HAI annually. These infections inevitably lead to increased medical costs ranging from $28.4 billion to $45 billion annually. The number of HAIs and resulting deaths are even greater in less developed countries.

Several factors contribute to the spread and acquisition of HAI. These factors include patient contact with medical devices, patient contact with furniture in the healthcare institution, the particular unit that a patient stays in, the amount of time a patient stays in the healthcare institution, clinical procedures undergone by the patient, and patient demographics, among other factors. Contact with bed structures may also be harmful to patients because mattresses and pillows are frequently contaminated by multi-resistant pathogens, thereby leading to cross-patient infections. Infections are also transmitted indirectly from one patient to another via healthcare workers.

Healthcare institutions have attempted to reduce the spread of HAI by limiting certain patients to certain areas of the healthcare institution (popularly known as “quarantine”). However, these attempts are not followed systematically for most patients. These methods are also not very comprehensive and do not significantly mitigate the risks of patients acquiring and/or spreading infections generally.

A need exists, therefore, for methods and apparatus that meaningfully reduce the spread of HAI among patients in a healthcare institution.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Embodiments of the present invention relate to methods and apparatus for assigning patients to beds to prevent the spread of HAI. By considering both a patient's ability to acquire infection and the patient's ability to spread infection, the patient can be assigned to a certain area (e.g., a bed) to reduce the transmission of HAI among patients. Additionally, embodiments of the present invention also consider the possibility that particular beds or the like may themselves facilitate the spread of HAI among patients. By preventing occurrences of HAI, higher quality healthcare, decreased deaths, and decreased medical costs can be realized.

In one aspect, embodiments of the present invention relate to a method for assigning patients to hospital beds. The method includes computing a first score for at least one patient, the first score representing the at least one patient's ability to acquire infection, computing a second score for the at least one patient, the second score representing the at least one patient's ability to spread infection, and assigning the at least one patient to a bed selected from a plurality of beds, the assignment based on at least the first score and the second score so as to reduce the possibility of the at least one patient acquiring an infection or spreading an infection.

In some embodiments of the method, the method further comprises displaying a report indicating the bed to which each of the at least one patient is assigned. In some embodiments of the method, the report comprises graphical depictions of at least one bed and at least one patient assigned to the at least one depicted bed.

In some embodiments of the method, the method further comprises retrieving infection profile information concerning the at least one patient from a database, and wherein computing the first score for the at least one person comprises computing the first score utilizing the retrieved infection profile information, and wherein computing the second score for the at least one person comprises computing the second score utilizing the retrieved infection profile information.

In some embodiments of the method, the method further comprises computing a third score and a fourth score for at least one bed from the plurality of beds, the third score representing the incremental chance of a person acquiring an infection from placement in that bed and the fourth score representing the incremental chance of a person spreading an infection from placement in that bed, and wherein assigning the at least one patient to a bed is also based on the third score and the fourth score. In some embodiments of the method, the method comprises retrieving bed profile information concerning the at least one bed from a database, and wherein computing the third score for the at least one bed comprises computing the third score utilizing the retrieved bed profile information, and wherein computing the fourth score for the at least one bed comprises computing the fourth score utilizing the retrieved bed profile information. In some embodiments of the method, the computing of the first score and the computing of the second score uses an algorithm derived from infection profile information from a database and the computing of the third score and the computing of the fourth score uses an algorithm derived from bed profile information from a database.

In some embodiments of the method, the computing of the first score, the computing of the second score, or both, assigns a higher score to at least one patient for at least one factor selected from the group consisting of infected status, antibiotic resistant status of an infection, and the status of the infection as a hospital acquired infection.

In some embodiments of the method, the computing of the third score, the computing of the fourth score, or both, assigns a higher score to at least one bed based on a history of patients having an infection occupying that bed.

In some embodiments of the method, the method further comprises storing a record of the assignment of the at least one patient to the at least one bed in a database.

In another aspect, embodiments of the invention relate to an apparatus for assigning patients to hospital beds. The apparatus comprises a processor, an interface, and computer executable instructions operative on said processor for: computing a first score for at least one patient, the first score representing the at least one patient's ability to acquire infection, computing a second score for the at least one patient, the second score representing the at least one patient's ability to spread infection, and assigning the at least one patient to a bed selected from a plurality of beds, the assignment based on at least the first score and the second score so as to reduce the possibility of the at least one patient acquiring an infection or spreading an infection.

In some embodiments of the apparatus, the apparatus further comprises computer executable instructions operative on said processor for displaying on the interface a report indicating the bed to which each of the at least one patient is assigned. In some embodiments of the apparatus, the report comprises graphical depictions of at least one bed and at least one patient assigned to the at least one depicted bed.

In some embodiments of the apparatus, the apparatus further comprises computer executable instructions operative on said processor for retrieving infection profile information concerning the at least one patient from a database, and wherein the computer executable instructions for computing the first score for the at least one person comprise computer executable instructions for computing the first score utilizing the retrieved infection profile information, and wherein the computer executable instructions for computing the second score for the at least one person comprise computer executable instructions for computing the second score utilizing the retrieved infection profile information.

In some embodiments of the apparatus, the apparatus further comprises computer executable instructions operative on said processor for computing a third score and a fourth score for at least one bed from the plurality of beds, the third score representing the incremental chance of a person acquiring an infection from placement in that bed and the fourth score representing the incremental chance of a person spreading an infection from placement in that bed, and wherein assigning the at least one patient to a bed is also based on the third score and the fourth score.

In some embodiments of the apparatus, the apparatus further comprises computer executable instructions operative on said processor for retrieving bed profile information concerning the at least one bed from a database, and wherein the computer executable instructions for computing the third score for the at least one bed comprise computer executable instructions for computing the third score utilizing the retrieved bed profile information, and wherein the computer executable instructions for computing the fourth score for the at least one bed comprises computer executable instructions for computing the fourth score utilizing the retrieved bed profile information.

In some embodiments of the apparatus, at least one of the computer executable instructions for computing the first score and the computer executable instructions for computing the second score use an algorithm derived from infection profile information from a database and wherein at least one of the computer executable instructions for the computing of the third score and the computer executable instructions for the computing of the fourth score comprise an algorithm derived from bed profile information from a database.

In some embodiments of the apparatus, at least one of the computer executable instructions for computing the first score and the computer executable instructions for computing the second score assign a higher score to at least one patient for at least one factor selected from the group consisting of infected status, antibiotic resistant status of an infection, and the status of the infection as a hospital acquired infection.

In some embodiments of the apparatus, at least one of the computer executable instructions for computing the third score and the computer executable instructions for computing the fourth score assign a higher score to at least one bed based on a history of patients having an infection occupying that bed.

In some embodiments of the apparatus, the apparatus further comprises computer executable instructions for storing a record of the assignment of the at least one patient to the at least one bed in a database.

In yet another aspect, embodiments of the invention relate to a computer readable medium containing computer-executable instructions for performing a method for assigning patients to hospital beds, the method comprising computing a first score for at least one patient, the first score representing the at least one patient's ability to acquire infection, computing a second score for the at least one patient, the second score representing the at least one patient's ability to spread infection, and assigning the at least one patient to a bed selected from a plurality of beds, the assignment based on at least the first score and the second score so as to reduce the possibility of the at least one patient acquiring an infection or spreading an infection.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

The invention and embodiments thereof will be better understood when the following detailed description is read in conjunction with the accompanying drawing figures:

FIG. 1 illustrates an apparatus for assigning patients to beds in accordance with one embodiment of the invention;

FIG. 2 displays various factors considered when assigning patients to beds in accordance with one embodiment of the invention;

FIG. 3 schematically illustrates the infection scoring unit 106 of FIG. 1 in accordance with one embodiment of the invention;

FIG. 4 schematically illustrates the bed assignment unit 108 of FIG. 1 in accordance with one embodiment of the invention;

FIG. 5 is a report summarizing possible patient-bed pairs in accordance with one embodiment of the invention;

FIG. 6 illustrates a graphical representation of patients being assigned to certain beds in accordance with one embodiment of the invention;

FIG. 7 illustrates a flowchart of a method of assigning patients to beds in accordance with one embodiment of the invention; and

FIG. 8 illustrates a flowchart of a method of assigning patients to beds in accordance with another embodiment of the invention.

In the drawings, like reference characters generally refer to corresponding parts throughout the different views. Elements are not necessarily drawn to scale, emphasis instead being placed on the principles and concepts of operation.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or apparatus. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

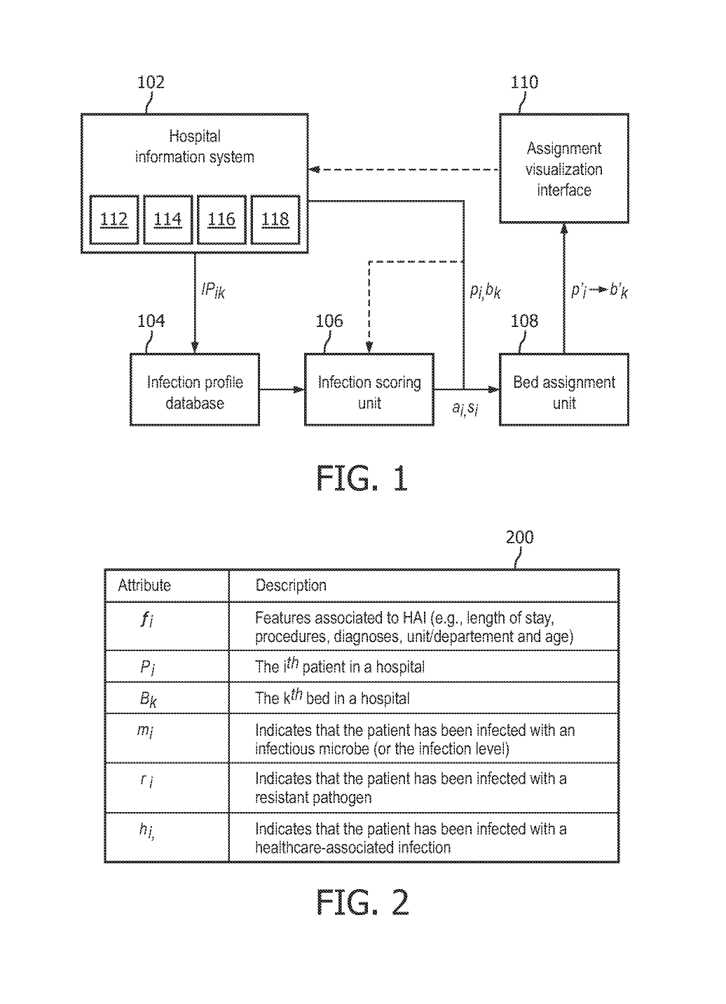

FIG. 1 generally illustrates an apparatus for assigning patients to beds in accordance with one embodiment of the invention. In this embodiment, the apparatus may include or otherwise be in communication with a hospital information system 102, an infection profile database 104, an infection scoring unit 106, a bed assignment unit 108, and an assignment visualization interface 110 to present information to medical personnel or other interested persons or systems.

The hospital information system 102 may store information related to patients, healthcare institution procedures, employees, or the like. The terms “hospital” and “hospital information system” are used for simplicity only, and the embodiments of the present invention are intended to include a variety of healthcare institutions. For example, the embodiments of the present invention may be implemented in areas such as doctor's offices, infirmaries, military compounds, urgent care institutions, academic institutions, or any other type of institution to prevent HAI. The hospital information system 102 may include one or more of a laboratory database 112, an administrative database 114, a demographics database 116, and an electronic health record database 118, among others.

The laboratory database 112 may store information relating to lab results regarding specific patients. This information may include results of tests performed on a certain patient, the location of where the test was performed, the locations of laboratories, or the like.

The administrative database 114 may store information relating to administrative tasks and other information related to the hospital. This information may include the number of admitted patients, the locations of each admitted patient, the number of open rooms in the hospital, the number of occupied/unoccupied beds in the hospital, information related to personnel shifts, appointments, and other types of administrative information.

The demographics database 116 may store information regarding patients or medical personnel. This may include information relating to the patient's age, sex, ethnicity, place of residency, whether the patient has traveled to a foreign country, or the like.

The electronic health record database 118 may include information regarding a patient's medical history. This information may include previous hospital visits, results of medical tests, patient blood types, whether a patient has allergies, whether a patient is currently affected (e.g., with a microbe, pathogen, HAI, etc.), or the like.

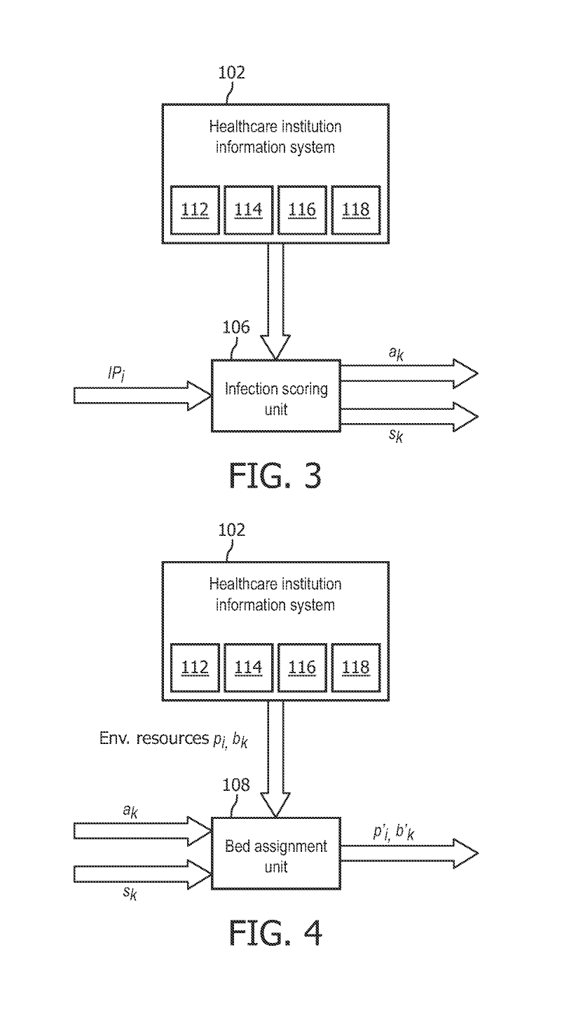

As illustrated in FIG. 3, the infection profile database 104 may be in operable communication with the hospital information system 102. This communication may be possible by any suitable wireless connection and/or a hardwired connection.

The infection profile database 104 may store infection-related information about inpatients as well as the beds in the hospital. A set of features fᵢ that are associated with HAI (e.g., length of patient stay in the hospital, procedures undertaken by the patient, test results, diagnoses, age, unit/department location, etc.) may be gathered from the hospital information system 102 and stored for each patient Pᵢ in the infection profile database 104. The infection profile database 104 may also store information related to each bed Bₖ that each patient Pᵢ uses in the hospital. For example, the infection profile database 104 may store information regarding whether a certain bed is or has been occupied by a patient with a pathogen.

These features fᵢ are used to develop one or more infection statuses. An infection status mᵢ indicates an infection is caused by an infectious microbe; an infection status rᵢ indicates an infection is caused by a resistant pathogen; and an infection status hᵢ indicates the infection is an HAI (i.e., the patient has acquired the infection during his/her stay at the healthcare institution). These attributes can be represented by the tuple {Pᵢ, Bₖ, fᵢ, mᵢ, rᵢ, hᵢ} and are summarized in table 200 of FIG. 2.

Referring back to FIG. 1, the attributes of the infection profile database 104 are communicated to the infection scoring unit 106. The infection scoring unit 106 may use novel algorithms to calculate scores representing the likelihood that a particular patient pᵢ may acquire an infection from another patient (i.e., an acquiring score aᵢ) or spread an infection to another patient (i.e., a spreading score sᵢ). The patient pᵢ may be a patient that has already been assigned to a bed or a new patient that has not yet been assigned to a bed (e.g., a patient who just entered the hospital). The infection scoring unit 106 may also develop acquiring and/or spreading scores for the beds bₖ (both free beds and occupied beds).

The infection scoring unit 106 can accept as an input a patient identifier (pᵢ), a bed identifier (bₖ), or both bed and patient identifiers (bₖ, pᵢ). If the input is a patient identifier pᵢ, the output may be an acquiring score aᵢ and/or a spreading score sᵢ. These outputs may be referred to as infection profiles, and essentially represent the probability that a particular patient pᵢ will acquire or spread an infection, respectively. To compute the acquiring score aᵢ, the infection scoring unit 106 may rely on an algorithm derived from infection profile information from the infection profile database 104, for example. A patient will tend to have a higher acquiring score aᵢ if they suffer from immune system-weakening diseases, have a history of acquiring infections, have features that match previous instances of HAI, and the like.

There are two main options for implementing the acquiring score algorithm: deterministic or empirical (data driven) models. There are well known epidemic models in the literature, such as the susceptible, infected and removed (SIR) model, which are based on deterministic equations. Using information from the infection profile as input, these models can be used to estimate the acquiring score aᵢ. Alternatively, empirical (data driven) models, such as those based on machine learning (k-Nearest Neighbors, Support Vector Machine, Neural Networks, etc.), can be used to estimate aᵢ. In this case, the machine learning algorithm may be trained so that the acquiring score aᵢ is learned (or estimated more accurately) for the set of input features fᵢ and a target class hᵢ.

Similarly, when assigning spreading scores sᵢ to a patient, the infection scoring unit 106 may consider whether the patient is known to be infected, whether the infection is caused by a drug-resistant infection, whether the infection was acquired in the hospital, and the like. To compute the spreading score sᵢ the infection scoring unit 106 may rely on an algorithm derived from bed profile information from the infection profile database 104, for example. Patients that are not infected are assigned low spreading scores sᵢ while patients with resistant HAI receive higher (infection) spreading scores sᵢ.

The method of calculating the spreading score sᵢ could follow the same approach as for calculating the acquiring score aᵢ, i.e., by using deterministic and/or empirical models. The difference will be in the empirical model, wherein the features fᵢ of a spreading infection score sᵢ for a patient and/or bed will be associated to a target class hᵢ′ of a patient/bed that acquired an infection and was in the same room as patient i.

If the input includes a bed identifier bₖ, the output may be a spreading score sₖ for the bed. This score may be based on, for example, the history of the patients that have been assigned to the bed and their infection scores. The more patients that have been assigned to a particular bed the higher the spreading score sₖ will tend to be, especially if those patients had high infection scores.

Typically acquiring scores aᵢ and/or spreading scores sᵢ are computed for at least a plurality of patients, and often for a plurality of beds to which those patients are to be assigned. The acquiring score(s) aᵢ and/or the spreading score(s) sᵢ may then be communicated to the bed assignment unit 108. The bed assignment unit 108 may be any specially configured processor or the like that receives as input the acquiring score(s) and/or spreading score(s) and determines a beneficial assignment for the associated patients that reduces the possibility that one or more of those patients will contract an HAI.

The bed assignment unit 108 is illustrated in FIG. 4 and may be in operable communication with the hospital information system 102 and the infection scoring unit 106 via any type of wireless connection or a hardwired connection. This connection provides the bed assignment unit 108 with real time and up-to-date information regarding resources at the hospital. This information may include the number of unoccupied beds, whether certain beds are occupied/unoccupied, the distances between each bed, as well as information regarding patients (e.g., their infection profiles) in the vicinity of these unoccupied beds, and the like. Additionally, medical personnel or other interested parties may input additional information for the bed assignment unit 108 to consider when assigning patients to beds.

The bed assignment unit 108 may use multiple-objective resource methods for assigning patients (p′ᵢ) to beds (b′ₖ) so that the probability of any particular patient acquiring an infection is at a minimum min aᵢ′ and that the probability of any particular patient spreading an infection is at a minimum min sᵢ′. The assignment(s) may be based on at least the infection scores of the already-assigned patients and their beds (both free and occupied beds). In further embodiments, the bed assignment unit 108 may consider a series of if/then rules, fuzzy logic, or the like when assigning patients to beds.

The multi-objective resource allocation algorithm can be implemented so that min(fₐ, fₛ,) where fₐ=Σᵢ₌₁ⁿaᵢ′ and fₛ=Σᵢ₌₁ⁿsᵢ′ for n=number of patients. This equation can be solved using several methods known in the literature including, but not limited to, scalarizing, no-preference, a priori and a posteriori methods, among others.

In use, for example, a patient who has gone through a surgery procedure will likely not be assigned to a bed that is immediately next to a patient infected with a resistant pathogen or assigned to a bed where a patient with a sepsis was previously located. Similarly, a patient with an acute lung infection would not be assigned immediately next to an immunosuppressed patient or to a patient taking immunosuppressant drugs if there are better bed assignment options available.

The output of the bed assignment unit 108 may be a list of combinations (p′ᵢ, b′ₖ) representing suitable patient-bed pairs. The bed assignment unit 108 may be configured to output only one pair (i.e., the most optimal pair), a list of any number of possible pairs in order of most optimal to least optimal, a list of pairs in order of least optimal to most optimal, or configured to present the pair(s) in some other way. The bed assignment unit 108 may also suggest assignments that would require already-assigned patients to be moved to different beds in order to create the most optimal patient-bed assignments.

The bed assignment unit 108 may also be configured to present a brief message regarding one or more proposed patient-bed pairs. For example, the message may explain why a certain proposed pair is more-preferable/less-preferable than another pair.

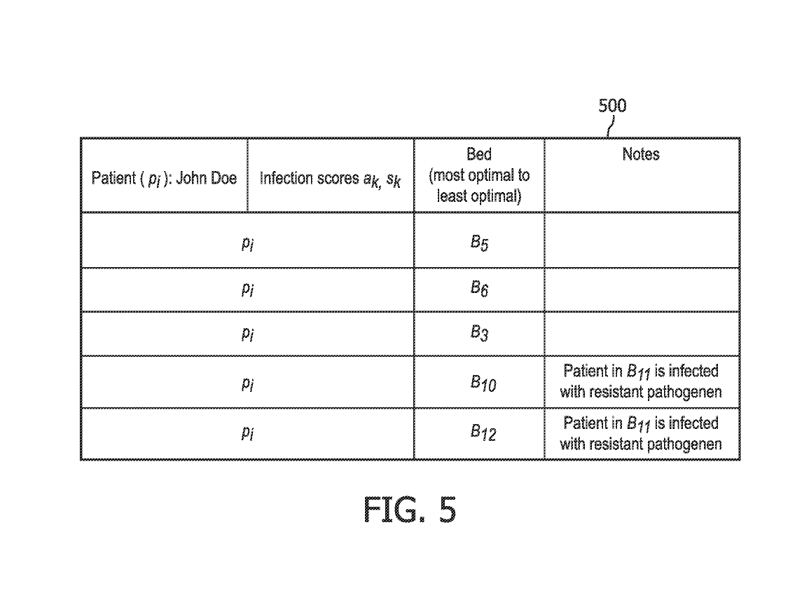

FIG. 5 illustrates an exemplary report 500 that may be the output of the bed assignment unit 108. The report 500 presents several potential bed assignments for a particular patient based on his/her infections score(s), ordered from most optimal (i.e., the assignment with the least probability of acquiring and spreading an infection) to the least optimal (which may still be a better result than a random assignment). As can generally be seen, placing this particular patient in bed B₅ would be the most optimal, while placing this patient in beds B₁₀ or B₁₂ would be the least optimal because there is a patient in B₁₁ that is infected with a resistant pathogen.

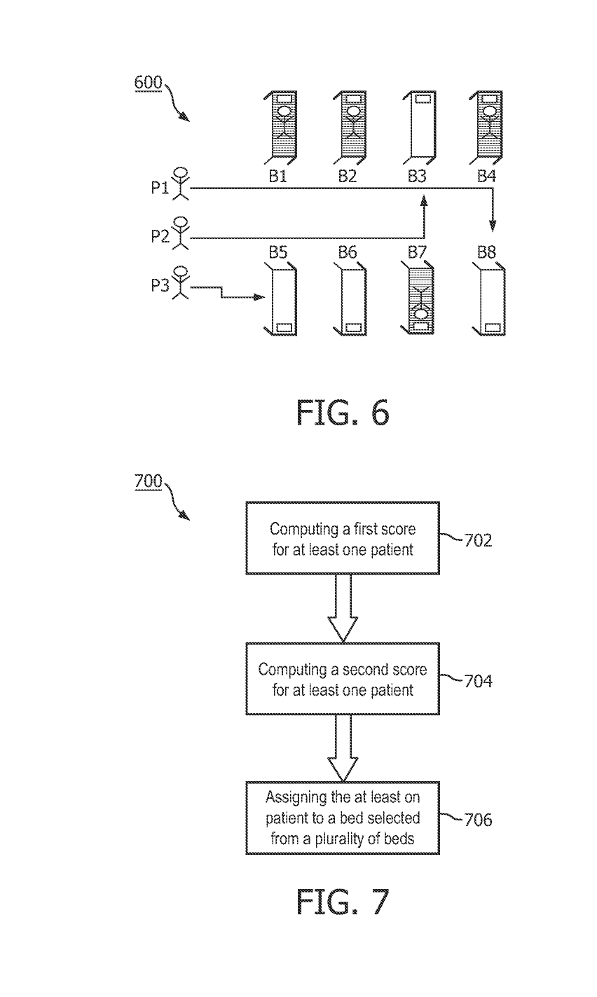

FIG. 6 illustrates an exemplary display 600 that can graphically illustrate the layout of a particular ward in the hospital and the proposed assignments for particular patients. In this particular instance, there are three patients P₁, P₂, and P₃ that each need to be placed in a bed in the ward. Based on their respective infection scores, the bed assignment unit 108 may determine the best locations for each of the patients based on the available beds and other environmental resources.

The outputs of the bed assignment unit 108 such as the report 500 or display 600 may be presented to the patient or medical personnel in a variety of ways. Referring back to FIG. 1, these outputs may be shown on the assignment visualization interface 110, which may be implemented as PC monitors, laptops, tablets, or any other type of display device. The output of the bed assignment unit 108 may be shared, emailed, printed, etc., and passed along to medical personnel such as in the ward itself to ensure proper placement of patients.

FIG. 7 illustrates a flowchart of a method 700 for assigning patients to hospital beds in accordance with one embodiment of the invention. Step 702 involves computing a first score for at least one patient. This first score may represent the at least one patient's ability to acquire an infection. As previously discussed, this acquiring score aᵢ may be determined by the infection scoring unit 106.

Step 704 of method 700 involves computing a second score for the patient. This second score may represent the patient's ability to spread an infection. As previously discussed, this spreading score sᵢ may be determined by the infection scoring unit 106. It is noted that the steps 702 and 704 may be performed simultaneously, contemporaneously, sequentially, etc.

Step 706 of method 700 involves assigning the patient(s) to a bed (or beds) selected from a plurality of beds. This assignment may be made by the bed assignment unit 108 and based on the first score and the second score to reduce the possibility of a patient acquiring an infection and/or spreading an infection. This method may be performed for each patient that needs to be assigned to a bed.

In some embodiments, multiple bed assignments may be made substantially contemporaneously at the same time to optimize the bed assignments for a plurality of patients. Such assignments may involve trade-offs, i.e., assigning a first patient to that patient's second best bed so that a more vulnerable second patient may be assigned to the bed that is best for that second patient.

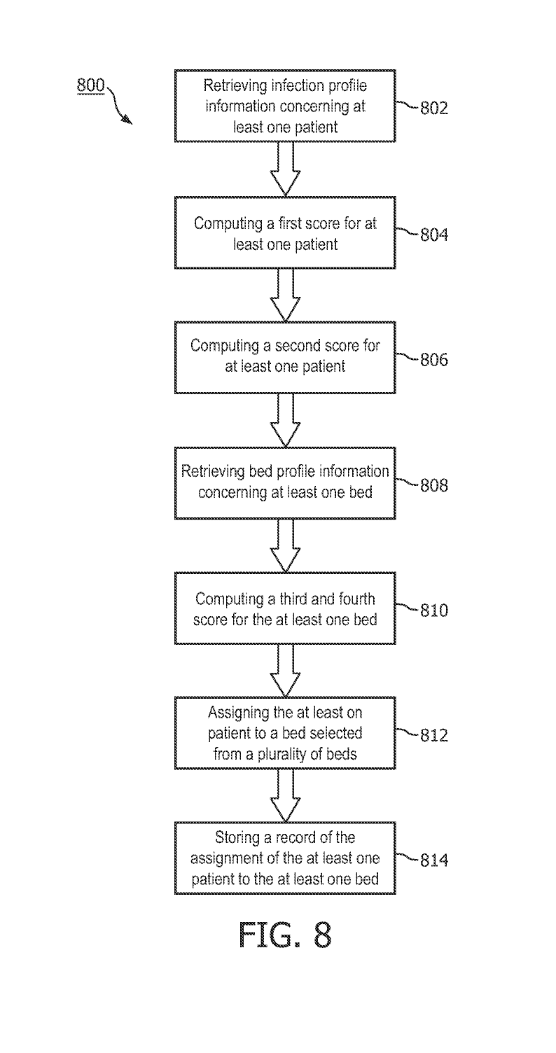

FIG. 8 generally illustrates a method 800 of assigning patients to hospital beds in accordance with another embodiment of the invention. Step 802 involves retrieving infection profile information concerning at least one patient from a database. This database may be the infection profile database 104 (and the hospital information system 102) discussed previously.

Step 804 involves computing a first score for the at least one patient. The first score may represent the patient's ability to acquire an infection and is based on at least the infection profile information retrieved in step 802. Several factors may be considered when computing the first score. These factors may include, but are not limited to, the patient's infected status, the antibiotic resistant status of an infection, and the status of the infection as a hospital acquired infection (HAI). As previously discussed, this acquiring score aᵢ may be determined by the infection scoring unit 106.

Step 806 involves computing a second score for the at least one patient. The second score may represent the patients' ability to spread an infection and is based on at least the infection profile information retrieved in step 802. As previously discussed, this spreading score sᵢ may be determined by the infection scoring unit 106.

Step 808 involves retrieving bed profile information concerning at least one bed. This information may be obtained from the hospital information system 102 and/or the infection profile database 104.

Step 810 involves computing a third score and a fourth score for at least one bed from the plurality of beds. The third score may represent the incremental chance of a person acquiring an infection from placement in a particular bed, and the fourth score may represent the incremental chance of a person spreading an infection from placement in that particular bed. As previously discussed, acquiring and spreading scores for the bed(s) may be determined by the infection scoring unit 106.

Step 812 involves assigning the at least one patient to a bed selected from a plurality of beds. This assignment may be based on the first, second, third, and fourth scores so as to reduce the possibility of the at least one patient acquiring an infection and spreading an infection.

Step 814 involves storing a record of the at least one patient to at least one bed. This record may be stored in a database such as in the hospital information system 102 and/or the infection profile database 104.

It is also noted that any of the steps of method 800 may be performed simultaneously, contemporaneously, sequentially, etc. As mentioned previously, outcomes of the patient assignments may be presented tabularly and/or graphically, for example.

In some embodiments, multiple bed assignments may be made substantially contemporaneously at the same time to optimize the bed assignments for a plurality of patients. Such assignments may involve trade-offs, i.e., assigning a first patient to that patient's second best bed so that a more vulnerable second patient may be assigned to the bed that is best for that second patient.

The features of the embodiments of the invention may implemented in a variety of environments. Specifically, these features may be implemented in clinical environments where patient (or user) data is collected via monitors and electronic records, and a patient or multiple patients need to be assigned to specific departments, wards or beds.

The methods, systems, and apparatus discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims. For example, the features of the invention may be implemented in hospitals, physician offices, military camps, infirmaries, urgent care facilities, maternity wards, or the like.

Although this application largely concerns assigning patients to appropriate beds, it is contemplated that other items may similarly be assigned to patients (or users in general). For example, individuals may be matched with certain rooms or general areas in an institution. Or, individuals may be assigned certain pieces of equipment (e.g., medical equipment) based on similar concerns and to prevent infections from spreading.

1. A method for assigning patients to hospital beds, the method comprising:
computing a first score for at least one patient, the first score representing the at least one patient's ability to acquire infection;
computing a second score for the at least one patient, the second score representing the at least one patient's ability to spread infection; and
assigning the at least one patient to a bed selected from a plurality of beds, the assignment based on at least the first score and the second score so as to reduce the possibility of the at least one patient acquiring an infection or spreading an infection.

2. The method of claim 1, the method further comprising displaying a report indicating the bed to which each of the at least one patient is assigned.

3. The method of claim 2, wherein the report comprises graphical depictions of at least one bed and at least one patient assigned to the at least one depicted bed.

4. The method of claim 1, further comprising retrieving infection profile information concerning the at least one patient from a database, and wherein computing the first score for the at least one person comprises computing the first score utilizing the retrieved infection profile information, and wherein computing the second score for the at least one person comprises computing the second score utilizing the retrieved infection profile information.

5. The method of claim 1, further comprising computing a third score and a fourth score for at least one bed from the plurality of beds, the third score representing the incremental chance of a person acquiring an infection from placement in that bed and the fourth score representing the incremental chance of a person spreading an infection from placement in that bed, and wherein assigning the at least one patient to a bed is also based on the third score and the fourth score.

6. The method of claim 5, further comprising retrieving bed profile information concerning the at least one bed from a database, and wherein computing the third score for the at least one bed comprises computing the third score utilizing the retrieved bed profile information, and wherein computing the fourth score for the at least one bed comprises computing the fourth score utilizing the retrieved bed profile information.

7. The method of claim 5, wherein at least one of the computing of the first score and the computing of the second score use an algorithm derived from infection profile information from a database and at least one of the computing of the third score and the computing of the fourth score uses an algorithm derived from bed profile information from a database.

8. The method of claim 1, wherein at least one of the computing of the first score and the computing of the second score assigns a higher score to at least one patient for at least one factor selected from the group consisting of infected status, antibiotic resistant status of an infection, and the status of the infection as a hospital acquired infection.

9. The method of claim 5, wherein at least one of the computing of the third score and the computing of the fourth assigns a higher score to at least one bed based on a history of patients having an infection occupying that bed.

10. The method of claim 1, further comprising storing a record of the assignment of the at least one patient to the at least one bed in a database.

11. An apparatus for assigning patients to hospital beds, the apparatus comprising:
a processor;
an interface; and
computer executable instructions operative on said processor for:
computing a first score for at least one patient, the first score representing the at least one patient's ability to acquire infection;
computing a second score for the at least one patient, the second score representing the at least one patient's ability to spread infection; and
assigning the at least one patient to a bed selected from a plurality of beds, the assignment based on at least the first score and the second score so as to reduce the possibility of the at least one patient acquiring an infection or spreading an infection.

12. The apparatus of claim 11, the apparatus further comprising computer executable instructions operative on said processor for displaying on the interface a report indicating the bed to which each of the at least one patient is assigned.

13. The apparatus of claim 12, wherein the report comprises graphical depictions of at least one bed and at least one patient assigned to the at least one depicted bed.

14. The apparatus of claim 11, further comprising computer executable instructions operative on said processor for retrieving infection profile information concerning the at least one patient from a database, and wherein the computer executable instructions for computing the first score for the at least one person comprise computer executable instructions for computing the first score utilizing the retrieved infection profile information, and wherein the computer executable instructions for computing the second score for the at least one person comprise computer executable instructions for computing the second score utilizing the retrieved infection profile information.

15. The apparatus of claim 11, further comprising computer executable instructions operative on said processor for computing a third score and a fourth score for at least one bed from the plurality of beds, the third score representing the incremental chance of a person acquiring an infection from placement in that bed and the fourth score representing the incremental chance of a person spreading an infection from placement in that bed, and wherein assigning the at least one patient to a bed is also based on the third score and the fourth score.

16. The apparatus of claim 15, further comprising computer executable instructions operative on said processor for retrieving bed profile information concerning the at least one bed from a database, and wherein the computer executable instructions for computing the third score for the at least one bed comprise computer executable instructions for computing the third score utilizing the retrieved bed profile information, and wherein the computer executable instructions for computing the fourth score for the at least one bed comprises computer executable instructions for computing the fourth score utilizing the retrieved bed profile information.

17. The apparatus of claim 15, wherein at least one of the computer executable instructions for computing the first score and the computer executable instructions for computing the second score use an algorithm derived from infection profile information from a database and wherein at least one of the computer executable instructions for the computing of the third score and the computer executable instructions for the computing of the fourth score comprise an algorithm derived from bed profile information from a database.

18. The apparatus of claim 11, wherein at least one of the computer executable instructions for computing the first score and the computer executable instructions for computing the second score assign a higher score to at least one patient for at least one factor selected from the group consisting of infected status, antibiotic resistant status of an infection, and the status of the infection as a hospital acquired infection.

19. The apparatus of claim 11, wherein at least one of the computer executable instructions for computing the third score and the computer executable instructions for computing the fourth score assign a higher score to at least one bed based on a history of patients having an infection occupying that bed.

20. The apparatus of claim 11, further comprising computer executable instructions for storing a record of the assignment of the at least one patient to the at least one bed in a database.

21. A computer readable medium containing computer-executable instructions for performing a method for assigning patients to hospital beds, the method comprising:
computing a first score for at least one patient, the first score representing the at least one patient's ability to acquire infection;
computing a second score for the at least one patient, the second score representing the at least one patient's ability to spread infection; and
assigning the at least one patient to a bed selected from a plurality of beds, the assignment based on at least the first score and the second score so as to reduce the possibility of the at least one patient acquiring an infection or spreading an infection.
